(19) United States
(12) Patent Application Publication
(10) Pub. No.: US 2026/0224772 A1
(43) Pub. Date: Aug. 6, 2026

(54) WOUND DRESSING WITH TISSUE REGENERATION EFFECT AND WOUND DRESSING PRODUCTION METHOD

(71) Applicant: AFYONKARAHISAR SAGLIK BILIMLERI UNIVERSITESI OZEL KALEM (REKTORLUK), Merkez, Afyonkarahisar (TR)

(72) Inventors: Feyza Sevim ERDOGMUS, Merkez, Afyonkarahisar (TR); Nurullah OKUMUS, Merkez, Afyonkarahisar (TR); Ozlem Erdal ALTINTAS, Merkez, Afyonkarahisar (TR); Sefa CELIK, Merkez, Afyonkarahisar (TR)

(21) Appl. No.: 19/158,376

(22) PCT Filed: Feb. 28, 2024
(86) PCT No.: PCT/TR2024/050164

§ 371 Date: Aug. 20, 2025

(30) Foreign Application Priority Data
Feb. 28, 2023 (TR) 2023/002231

(51) Int. Cl. A61L 15/28; A61F 13/00; A61F 13/01; A61L 15/44; C08L 5/08

(52) U.S. Cl. CPC A61L 15/28; A61F 13/00987; A61F 13/01012; A61F 13/01046; A61L 15/44; C08L 5/08; A61F 2013/00157; A61F 2013/00255; A61L 2400/12; A61L 2430/00

(57) ABSTRACT

A wound dressing with tissue regeneration effect for medical use in the treatment of wounds and burns, which provides protection of the damaged tissue area from external influences and absorption of the damaged tissue fluid, as well as enhancement of tissue regeneration in the damaged tissue area, controlled release of bioactive components in the plant extract, reduction of pore size, while at the same time increasing water holding capacity and air permeability to accelerate healing of the damaged tissue, and a method of production of this wound dressing.

### TECHNICAL FIELD

The invention relates to a wound dressing with tissue regeneration effect and a method of manufacturing the same, which allows for increased tissue regeneration efficiency, controlled release of bioactive components, increased water holding capacity and air permeability while reducing pore size.

In particular, the invention relates to a wound dressing with tissue regeneration effect for medical use in the treatment of wounds and burns, which provides protection of the damaged tissue area from external influences and absorption of the damaged tissue fluid, as well as enhancement of tissue regeneration in the damaged tissue area, controlled release of bioactive components in the plant extract, reduction of pore size, while at the same time increasing water holding capacity and air permeability to accelerate healing of the damaged tissue, and a method of production of this wound dressing.

### STATE OF THE ART

In the treatment of wounds and/or burns, materials that are designed to provide suitable environmental conditions for the healing of the damaged tissue by covering the damaged tissue area, providing an artificial tissue scaffold for cells and hygienic conditions are called wound dressings. Wound dressings are materials that protect the wounded area against microorganisms and possible external factors to prevent the formation of infection, absorb blood and inflammation fluid and provide wound healing by releasing drugs when desired. An ideal wound dressing is expected to reduce heat loss, prevent water loss, reduce protein loss due to exudate (fluid leaking into a body cavity as a result of injury or inflammation) in burn tissue, prevent interaction with microorganisms, make the patient feel less pain in dressing changes, allow painless movement, help tissue formation for skin regeneration, accelerate wound healing in superficial burns and be biocompatible.

Traditional wound/burn dressings are produced from natural, synthetic or semi-synthetic materials to protect the wound area from external influences and to absorb wound fluid. While classifying these dressings, many parameters such as material content, dressing technique and wound type are taken into consideration. The main functions of traditional wound dressings are to keep the wound dry and warm, to physically protect the wound site and to provide absorption of wound exudates. Generally, their inner parts consist of cellulose filler or cotton fibers, while their outer layers contain polyamide and whiskey fibers. By adding materials such as polyamide contact layer, viscose fibers coated with polypropylene to traditional wound dressings such as bandages, non-woven felts, absorbent tampons, etc., these wound dressings are given properties such as low adhesion, easy removal from the wound, etc. and their general usage properties are improved. Despite their advantages such as high absorption capacity and partial air permeability, thermal insulation, etc., the biggest problem with these wound dressings is that they lack the necessary moist surface for the wound. This causes the wound/burn area to dry out, delaying epithelialization, which is formed by the proliferation of epithelial cells in the damaged area, which provides healing. In addition, with the disruption of fiber integrity in fibrous structures such as gauze, some microorganisms can easily enter the wound and this causes infection in the damaged area.

Delivery of drugs or drug substances to the target site is one of the main problems in pharmaceutical and biotechnological fields, especially in wound and burn treatment. Failure of the transported drugs or drug substances to show their effects within the specified time or to show their effects in the whole body other than the target tissue leads to undesirable situations. Drug delivery refers to formulations prepared to safely transport pharmaceutical ingredients to the needed area in the body. The definition of drug delivery system is used for a material, system or device that aims to deliver therapeutic agent or agents to where they are needed in the body and enables the release of these agents over time. These systems are designed to regulate or modify the biodistribution and pharmacokinetics (the processes of absorption, distribution, biotransformation, elimination, etc. of drugs in the body) of the drug loaded into their structure. However, these systems must be compatible with the drug, allow drug loading at desired dosages, and be suitable for easy removal from the body at the end of treatment. The primary goal in pharmaceutical studies is to minimize the dosage of the drug/active substance and improve the patient's quality of life. Controlled release systems are best able to meet these expectations and offer many advantages. These include maintaining the level of drug substance at a therapeutic level, targeting the release to a specific cell or tissue, reducing the amount of drug or drug substance needed, facilitating the administration of drugs or drug substances with short half-lives, and keeping the drug or drug substance at a specific site for the desired duration. However, conventional drug delivery systems, which are taken into the body through the oral route, intravenous route or inhalation, have a certain efficacy in the body, but are not efficient due to their undesirable side effects, low degradation and transient efficacy. Since many drugs taken into the body have systemic rather than local effects, the occurrence of undesirable side effects limits the use of these systems. In addition, drugs used with this system cannot reach an effective concentration in the body due to hydrolysis, enzymatic degradation, pH changes, etc. or maintain their effectiveness in the damaged area for a long time.

A patent document U.S. Pat. No. 10,293,011B2 in the known state of the art discloses a chemical composition comprising natural substances with healing, repellent and biocidal properties for the treatment and healing of wounds on the external surface of the body. The said composition is suitable for use in human and animal treatment and contains neem oil mixed with Hypericum oil extract. The composition provides anti-bacterial, anti-inflammatory and analgesic properties, as well as repellent/repellent action against myiasigenic diptera and non-myiasigenic diptera. The document does not mention a solution for enhancing tissue regeneration in the damaged tissue area, controlled release of bioactive components in the plant extract, reducing pore size while at the same time increasing water holding capacity and air permeability to accelerate healing of the damaged tissue.

Another patent document KR100740596B1 of the known art discloses a method of manufacturing an adhesive band-aid used in the treatment of cuts and burns, which allows the polymer hydrogel to adhere easily to the tissue and detach from the tissue without causing discomfort. In the method mentioned, the liquid polymer solution is temporarily added to the adhesive surface of the bandage and then sterilized by cross-linking. In this way, a hydrogel-containing wound dressing that adheres with the effect of pressure is obtained and the production method eliminates the drying and skin irritation problem in other hydrogel-containing tapes. The document does not mention a solution for enhancing tissue regeneration in the damaged tissue area, controlled release of bioactive components in the plant extract, reducing pore size while at the same time increasing water holding capacity and air permeability to accelerate healing of the damaged tissue.

As a result, there is a need for the development of a wound dressing with tissue regeneration effect and its production method, which is suitable for medical use in wound and burn treatment, which protects the damaged tissue area from external influences and increases tissue regeneration in the damaged tissue area as well as absorption of damaged tissue fluid, controlled release of bioactive components in the plant extract, reduction of pore size, water retention capacity, air permeability and healing rate of damaged tissue.

### PURPOSE OF THE INVENTION

The present invention relates to a wound dressing with tissue regeneration effect and a method of manufacturing a wound dressing with tissue regeneration effect, which fulfills the above-mentioned requirements, eliminates possible disadvantages and provides some additional advantages.

The main purpose of the inventive wound dressing with tissue regeneration effect and wound dressing production method is to obtain a wound dressing with tissue regeneration effect and wound dressing production method that provides protection of the damaged tissue area from external influences, drainage and absorption of damaged tissue fluid and enhancement of tissue regeneration in the damaged tissue area.

Another object of the invention is to provide a wound dressing with tissue regeneration effect and a method of manufacturing a wound dressing, which enables the release of bioactive components in the plant extract to be carried out in a controlled manner and the effectiveness of tissue regeneration to be increased.

Another object of the invention is to provide an effective and efficient wound dressing with tissue regeneration effect and wound dressing production method, which enables the pore size to be reduced, water retention capacity, air permeability and healing speed of the damaged tissue to be increased.

Another object of the invention is to provide a practical and functional wound dressing with tissue regeneration effect and wound dressing production method that prevents the penetration of exogenous bacteria into the damaged tissue and protects the damaged tissue against dehydration.

Another object of the invention is to provide a wound dressing with tissue regeneration effect and a method of manufacturing a wound dressing, which enables to obtain a flexible, size-adjustable and non-woven form, thereby increasing the diversity of use and protection effectiveness.

Another object of the invention is to provide a wound dressing with tissue regeneration effect and a method of manufacturing a wound dressing, which enables the placement of therapeutic agents in different layers by including multiple layers and allows the production of multiple layers together to include agents placed in different layers.

An alternative aim of the invention is to obtain an effective wound dressing with effective tissue regeneration and dressing production method, which reduces the scar and scar tissue remaining after healing in the damaged tissue.

In order to achieve the above objectives in the most general form, it contains at least one first bioactive component suitable for use on the damaged tissue area, providing Wound dressing with tissue regeneration effect that protects the damaged tissue area from external influences, absorbs damaged tissue fluid, increases tissue regeneration, provides controlled release of bioactive components, increases water holding capacity and air permeability while reducing pore size, prevents the passage of bacteria and infection-causing substances into the damaged tissue and accelerates the healing process, at least one medical matrix containing fungal-derived chitosan in nanofiber structure, enhancing the effectiveness of tissue regeneration and controlled release of bioactive components, at least one medical matrix that physically supports cells and provides cell adhesion, Hypericum perforatum in nanoparticle structure integrated with the medical matrix, enhancing tissue regeneration and healing effects, treating damaged tissue and bacterial and viral infection, and at least one second bioactive component containing Cistus laurifolius, which is in the structure of nanoparticles integrated with the medical matrix, increasing the healing efficiency, providing antimicrobial, antioxidant and antiviral effects by forming a physical defense layer against bacteria and viruses on the damaged tissue.

A method of manufacturing a wound dressing with tissue regeneration effect developed with the present invention, which enables the production of a wound dressing with tissue regeneration effect, includes the steps of, inoculation of fungi from agar medium to broth medium; incubation and biomass production followed by filtration and drying; precipitation and deacetylation to obtain chitosan from biomass and precipitation of chitosan; drying and grinding of Hypericum perforatum and Cistus laurifolius plants; extraction of ground plants in ultrasonic bath; filtration and collection of extracts; concentrating the extracts in a rotary evaporator; freeze-drying the extracts into powder; obtaining fungus-derived chitosan nanofibers by electrostatic spinning; obtaining nanoparticles from plant extracts using ultrasonics and integrating the particles into chitosan nanofibers.

The structural and characteristic features and all advantages of the invention will be more clearly understood by means of the figures given below and the detailed description written by making references to these figures, and therefore, the evaluation should be made by considering these figures and detailed description.

### FIGURES TO HELP UNDERSTAND THE INVENTION

In order to best understand the structure and advantages of the present invention, it should be evaluated together with the figures described below.

FIG. 1: A front view of the chitosan obtained by using a scanning electron microscope after the step of obtaining fungus-derived chitosan nanofibers in the production method of wound dressing with tissue regeneration effect subject to the invention.

FIG. 2: Another view of the nanoliposomes loaded with plant extracts after the step of obtaining nanoparticles from plant extracts in the production method of wound dressing with tissue regeneration effect subject to the invention using a scanning electron microscope.

FIG. 3: Another view of the nanoliposomes loaded with plant extract after the step of obtaining nanoparticles from plant extracts in the wound dressing with tissue regeneration effect production method subject to the invention using a scanning electron microscope.

FIG. 4: Another view of the nanofibers obtained by using a scanning electron microscope after the step of obtaining fungus-derived chitosan nanofibers by applying electrostatic spinning process in the wound dressing with tissue regeneration effect production method subject to the invention.

FIG. 5: Another view of the nanofibers taken using a scanning electron microscope after the step of integration of the nanoparticles obtained in the wound dressing with tissue regeneration effect production method, which is the subject of the invention, into chitosan nanofibers.

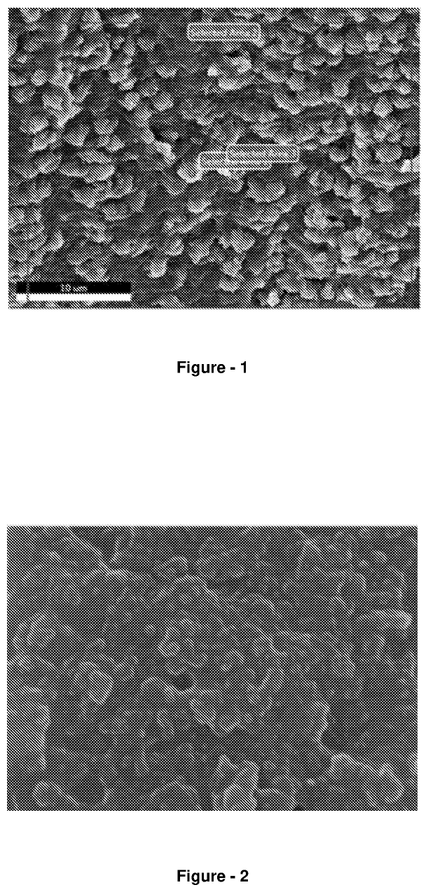

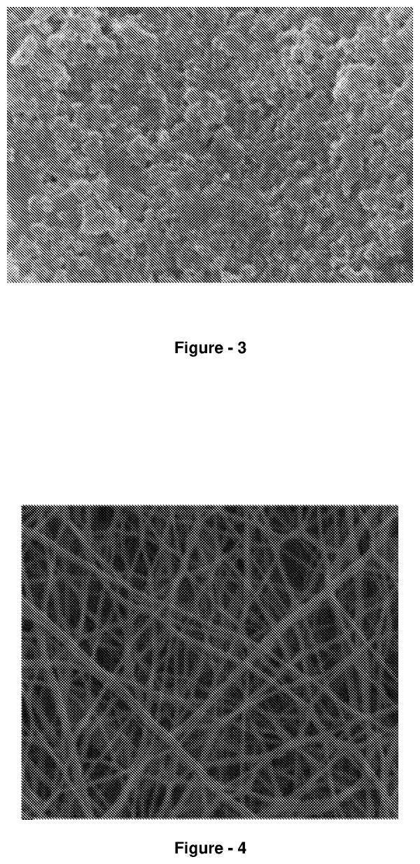

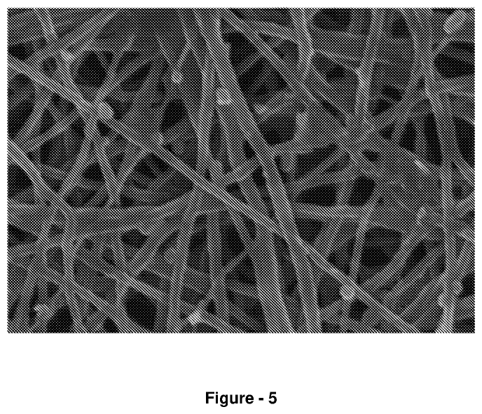

### DETAILED DESCRIPTION OF THE INVENTION

In this detailed description, the wound dressing with tissue regeneration effect and preferred embodiments of the wound dressing with tissue regeneration effect production method, which are the subject of the invention, are explained only for a better understanding of the subject and in a way that does not create any limiting effect.

Developed with the present invention, preferably suitable for use on a damaged tissue site, preferably wound or burn tissue, and in the most general form, contains at least one first bioactive component suitable for use as a therapeutic agent, for use on the damaged tissue area, providing biocompatible and biodegradable wound dressing with tissue regeneration effect that protects the damaged tissue area from external influences, absorbs damaged tissue fluid in the healing process of damaged tissue, increases tissue regeneration, preferably suitable for production by electro-spinning method, providing controlled release of bioactive components, increases water holding capacity and air permeability while reducing pore size, prevents the passage of bacteria and infection-causing substances into the damaged tissue and accelerates the healing process, at least one medical matrix containing fungal-derived chitosan in nanofiber structure, enhancing the effectiveness of tissue regeneration and controlled release of bioactive components, at least one medical matrix that physically supports cells and provides cell adhesion, Hypericum perforatum (St. John's wort) in nanoparticle structure integrated with the medical matrix, enhancing tissue regeneration and healing effects, treating damaged tissue and bacterial and viral infection, and at least one second bioactive component, preferably suitable for use as a therapeutic agent, containing Cistus laurifolius, which is in the structure of nanoparticles integrated with the medical matrix with the polyphenols in, increasing the healing efficiency, providing antimicrobial, antioxidant and antiviral effects by forming a physical defense layer against bacteria and viruses on the damaged tissue.

In an exemplary application of the wound dressing with tissue regeneration effect developed with the present invention, the wound dressing is laid on a damaged tissue area such as a wound and/or burn. Thus, the damaged area is protected against external influences, while the damaged tissue fluid is absorbed and the covered area is kept hygienic. Through the nanofiber form of the medical matrix in the wound dressing, the pore size in the porous structure is reduced and the pore density is increased. This prevents the penetration of bacteria and microbes into the wound or burn area, provides flexible, formable and non-woven production, allows the damaged tissue to breathe to accelerate healing and at the same time effectively absorbs tissue fluid. Through fact that the medical matrix consists of fungal-derived chitosan, a biocompatible, biodegradable and non-allergenic wound dressing is obtained. The first bioactive component integrated into the medical matrix, Hypericum perforatum (St. John's wort) in nanoparticle structure, enhances tissue regeneration and healing effects, and treats damaged tissue and bacterial and viral infection with its antioxidant effect. Meanwhile, the second bioactive component integrated into the medical matrix, Cistus laurifolius in the nanoparticle structure it contains, increases the healing efficiency of damaged tissue, and the polyphenols in Cistus laurifolius provide antimicrobial and antiviral effects by creating a physical defense layer against bacteria and viruses on the damaged tissue.

A method of manufacturing a wound dressing with tissue regeneration effect, enabling the production of a tissue regeneration effective wound dressing developed with the present invention includes preferably the steps of inoculation of fungi containing Rhizopus oryzae (NRRL 1526) and/or Aspergillus brasiliensis (ATTC 16404) from agar medium to broth medium; incubation of the obtained fungi, preferably at 27 0C, followed by filtration and drying after biomass production; precipitation and deacetylation to obtain chitosan from biomass and precipitation of chitosan; drying and grinding of Hypericum perforatum and Cistus laurifolius plants; extraction of the ground plants in an ultrasonic bath, preferably at 37 0C; collection of the extracts by filtration, preferably using filter paper; concentrating the plant extract in a rotary evaporator; drying the extracts in a freeze dryer (lyophilizer) and pulverizing them; obtaining fungus-derived chitosan nanofibers by electrostatic spinning; obtaining nanoparticles from plant extracts using ultrasonic method and—obtaining nanoparticles from plant extracts using ultrasonic method and integrating the obtained nanoparticles into chitosan nanofibers preferably using the nozzle system of electrostatic spinning device.

For example, although various methods in the literature have been tried for nanoparticle integration into chitasone-based nanofibers, these attempts have not been successful. Nanoparticles are integrated into the nanofiber and verified by SEM analysis with a method that is found in the literature but does not give positive results in trials. The method developed by the present invention utilizes an electro-spinning device for producing nanofibers. After the obtained nanofibers are placed on the drum/drum part of the device, the solution containing herbal nanoparticles is sprayed evenly on the nanofiber surface through the nozzle/nozzle system of the device at low voltage, at a spinneret distance of 100 mm, with a flow rate of 0.30 mL/s. Afterwards, the nanofiber is left to dry, followed by a washing process and then dried again. Then, whether the nanoparticles are integrated into the nanofibers is determined by SEM analysis. The release profiles of plant extracts from these nanofibers are extracted and controlled release is determined. The fact that the obtained biopolymer is non-toxic and biocompatible, has no side effects, and the plants used have natural ingredients makes it more advantageous than existing wound healers when the chemical contents of existing wound healers are considered.

Through the wound dressing with tissue regeneration effect and wound dressing with tissue regeneration effect production method developed with the present invention, it is ensured that the damaged tissue area is protected from external effects, the absorption of damaged tissue fluid is increased, as well as the tissue regeneration in the damaged tissue area is increased and the release of bioactive components in the plant extract is carried out in a controlled manner. Also, a safe, effective, practical and healthy wound dressing with tissue regeneration effect and its production method, which is suitable for medical use in wound and burn treatment, which reduces the penetration of bacteria and microbes by reducing the pore size, increases the water retention capacity, air permeability and healing speed of the damaged tissue by increasing the number of pores, and offers protection against bacteria, viruses and infection-causing substances, is obtained.

1. A biocompatible and biodegradable wound dressing with tissue regeneration effect, which is suitable for use on a damaged tissue area, protecting the damaged tissue area from external influences, absorbing damaged tissue fluid, increases tissue regeneration in the damaged tissue area and the release of bioactive components in a controlled manner and the water holding capacity and air permeability while reducing the pore sizes in the structure, prevents the passage of bacteria and infection-causing substances into the damaged tissue and accelerating the healing process of the damaged tissue, the wound dressing comprising:
at least one medical matrix comprising fungal-derived chitosan, wherein the fungal-derived chitosan is the form of nanofiber, which increases the efficiency of tissue regeneration in the damaged tissue area and controlled release of bioactive components, physically supports the cells in the healing process of the damaged tissue and provides adhesion of the cells by acting as a scaffold;
at least one first bioactive component comprising Hypericum perforatum, wherein the Hypericum perforatum is in the form of nanoparticles integrated with the medical matrix, which enhances tissue regeneration and healing effects, treats damaged tissue, bacterial and viral infection with its antioxidant effect; and
at least one second bioactive component comprising Cistus laurifolius, wherein the Cistus laurifolius is in the form of nanoparticles integrated with the medical matrix, which increases the healing efficiency of damaged tissue, provides antimicrobial, antioxidant and antiviral effect by creating a physical defense layer against bacteria and viruses on the damaged tissue with the polyphenols in it.

2. The wound dressing with tissue regeneration effect of claim 1, wherein the damaged tissue area is scar and/or burn tissue.

3. The wound dressing with tissue regeneration effect of claim 1, wherein the medical matrix is suitable for production by electro-spinning method.

4. The wound dressing with tissue regeneration effect of claim 1, wherein the first bioactive component is suitable for use as a therapeutic agent.

5. The wound dressing with tissue regeneration effect of claim 1, wherein the second bioactive component is suitable for use as a therapeutic agent.

6. A method of producing a wound dressing with tissue regeneration effect according to claim 1, the method comprising the steps of:
planting fungi from agar medium to broth medium;
applying incubation process to the fungi obtained and applying filtration and drying processes after biomass production;
obtaining chitosan from biomass and precipitating chitosan by applying precipitation and deacetylation processes;
drying and grinding Hypericum perforatum and Cistus laurifolius plants;
extracting the ground herbs in an ultrasonic bath;
collecting the extracts by filtration;
concentrating the plant extract in the rotary evaporator;
drying the extracts in a freeze dryer and pulverizing them;
obtaining fungus-derived chitosan nanofibers by applying electrostatic spinning process; and
obtaining nanoparticles from plant extracts using ultrasonic method and integrating the obtained nanoparticles into chitosan nanofibers.

7. The method according to 6, wherein the fungi contain Rhizopus oryzae and/or Aspergillus brasiliensis.

8. The method according to 6, wherein the incubation process is executed at a temperature of 27° C.

9. The method according to 6, wherein the extraction process is executed at a temperature of 37° C.

10. The method according to 6, comprising the step of filtering the extracts by using filter paper.

11. The method according to 6, comprising the step of integrating the obtained nanoparticles into chitosan nanofibers by using the nozzle system of the electrostatic spinning device.
